(19) United States
(12) Patent Application Publication
(10) Pub. No.: US 2023/0321406 A1
(43) Pub. Date: Oct. 12, 2023

(54) VASCULAR ACCESS DEVICE HAVING A PHOSPHORESCENT FEATURE

(71) Applicant: Becton, Dickinson and Company, Franklin Lakes, NJ (US)

(72) Inventor: Megan Scherich, Salt Lake City, UT (US)

(21) Appl. No.: 17/714,820

(22) Filed: Apr. 6, 2022

(51) Int. Cl. A61M 25/06; A61M 25/09; A61M 39/10; A61M 25/00

(52) U.S. Cl. CPC A61M 25/0637; A61M 25/0905; A61M 39/10; A61M 25/0045; A61M 2205/587; A61M 2039/1083; A61M 2205/0238; A61M 2207/10

(57) ABSTRACT

A catheter assembly may include a catheter hub, which may include a distal end, a proximal end, a lumen extending through the distal end and the proximal end, and a side port disposed between the distal end and the proximal end. The catheter assembly may also include a catheter extending distally from the catheter hub, an extension set coupled to the catheter hub, a catheter hub stabilization device coupled to the catheter hub, a needle hub coupled to the proximal end of the catheter hub, and an introducer needle coupled to the needle hub and extending through the catheter. One or more of the extension set, the catheter hub, or the catheter hub stabilization device may be phosphorescent. An instrument advancement device or a syringe may include one or more phosphorescent features.

### BACKGROUND

A common type of catheter assembly includes a peripheral intravenous catheter (“PIVC”) that is over-the-needle. As its name implies, the PIVC that is over-the-needle may be mounted over an introducer needle having a sharp distal tip. The catheter assembly may include a catheter hub, the PIVC extending distally from the catheter hub, and the introducer needle extending through the PIVC. The PIVC and the introducer needle may be assembled such that the distal tip of the introducer needle extends beyond the distal tip of the PIVC with the bevel of the needle facing up away from skin of the patient immediately prior to insertion into the skin. The PIVC and the introducer needle are generally inserted at a shallow angle through the skin into a blood vessel of the patient.

In order to verify proper placement of the introducer needle and/or the PIVC in the blood vessel, a clinician may confirm that there is flashback of blood in a flashback chamber of the catheter assembly. In some instances, blood may travel into the introducer needle and then out of a flashback notch in the introducer needle to reach the flashback chamber, where the blood is visible to the clinician. Once placement of the introducer needle has been confirmed by observation of the blood, the clinician may remove the introducer needle, leaving the PIVC in place in the blood vessel for future blood withdrawal or fluid infusion.

In order to perform a blood draw or infusion with the PIVC, a clinician may turn on a light in a patient's room, disturbing or waking up the patient. Such light exposure alters the release of melatonin in the body and may make it difficult for the patient to fall back asleep after being woken up. Disruption of the patient's sleep cycle may impair the patient's recovery because sleep cycles are critical for healing.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to a vascular access device having one or more phosphorescent features. In some embodiments, the vascular access device may include a catheter assembly. In some embodiments, the catheter assembly may include a catheter hub, which may include a distal end, a proximal end, a lumen extending through the distal end and the proximal end, and a side port disposed between the distal end and the proximal end.

In some embodiments, the catheter assembly may include a septum disposed within the lumen of the catheter hub and configured to prevent fluid leakage out of the catheter hub. In some embodiments, the catheter assembly may include a catheter extending distally from the catheter hub. In some embodiments, the catheter may include a peripheral intravenous catheter (“PIVC”), a peripherally-inserted central catheter (“PICC”), a midline catheter, an arterial catheter, a central venous catheter (“CVC”) or another suitable catheter.

In some embodiments, the catheter assembly may include an extension set coupled to the catheter hub. In some embodiments, the catheter assembly may include a catheter hub stabilization device coupled to the catheter hub. In some embodiments, the catheter assembly may include a needle hub coupled to the proximal end of the catheter hub. In some embodiments, the catheter assembly may include an introducer needle coupled to the needle hub and extending through the catheter. In some embodiments, the extension set, the catheter hub, or the catheter hub stabilization device may be phosphorescent.

In some embodiments, the extension set may extend proximally from the side port and may include an extension tube and a luer adapter disposed at a proximal end of the extension tube. In some embodiments, the luer adapter may be phosphorescent. In some embodiments, the extension set may extend proximally from the side port, and the extension set may include the extension tube and a clamp disposed on the extension tube. In some embodiments, the clamp is phosphorescent. In some embodiments, the luer adapter and the clamp may be phosphorescent.

In some embodiments, the extension set may extend proximally from the side port. In some embodiments, the extension set may include the extension tube and the luer adapter disposed at a proximal end of the extension tube, and the extension set may further include another extension tube extending from the side port and coupled to another luer adapter. In some embodiments, the extension tube may extend from the other luer adapter. In some embodiments, the other luer adapter may be phosphorescent.

In some embodiments, the distal end of the catheter hub may be phosphorescent. In some embodiments, the catheter stabilization device may be phosphorescent. In some embodiments, the catheter stabilization device may include a wing extending outwardly from the catheter hub. In some embodiments, the wing may be phosphorescent. In some embodiments, the catheter stabilization device may include a platform selectively coupled to the catheter hub. In some embodiments, the platform may be phosphorescent.

In some embodiments, the vascular access device may include an instrument advancement device, which may be configured to advance an instrument distally through the catheter assembly. In some embodiments, the instrument advancement device may include a housing, which may include a slot. In some embodiments, the instrument advancement device may include an advancement tab disposed within the slot. In some embodiments, the instrument advancement device may include an instrument disposed within the housing and coupled to the advancement tab. In some embodiments, the instrument may include a guidewire or a catheter.

In some embodiments, the instrument advancement device may include a distal connector configured to coupled to the catheter assembly. In some embodiments, the instrument may include a guidewire or a catheter. In some embodiments, the instrument advancement device may include a support feature disposed within the housing and configured to reduce buckling of the instrument. In some embodiments, the support feature, the advancement tab, or the distal connector may be phosphorescent. In some embodiments, the advancement tab may be phosphorescent. In some embodiments, the distal connector may be phosphorescent. In some embodiments, the support feature may be phosphorescent.

In some embodiments, the vascular access device may include a syringe configured to be coupled to a catheter assembly to draw blood and/or flush the catheter assembly. In some embodiments, the syringe may include a barrel, which may include a distal end configured to couple to the catheter assembly. In some embodiments, the syringe may include a plunger configured to depress within the barrel. In some embodiments, the syringe may include a phosphorescent feature, which may include a phosphorescent material molded into the barrel or a sticker on the barrel comprising phosphorescent text indicating a volume. In some embodiments, the phosphorescent feature comprises the sticker, and the phosphorescent text may include multiple numbers.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 is an upper perspective view of an example catheter assembly, illustrating an example phosphorescent extension set, according to some embodiments;

FIG. 2 is an upper perspective view of an example stabilization device configured to be selectively coupled to a catheter hub, illustrating another example phosphorescent extension set, according to some embodiments;

FIG. 3 is an upper perspective view of the catheter assembly, illustrating an example phosphorescent stabilization device and an example phosphorescent catheter hub, according to some embodiments;

FIG. 4 is an upper perspective view of the stabilization device configured to be selectively coupled to a catheter hub, illustrating an example phosphorescent platform, according to some embodiments;

FIG. 5 is an upper perspective view of an example instrument advancement device, according to some embodiments;

FIG. 6 is an upper perspective view of an example syringe, according to some embodiments; and

FIG. 7 is an upper perspective view of an example phosphorescent dressing placed on an arm of a patient, according to some embodiments.

### DESCRIPTION OF EMBODIMENTS

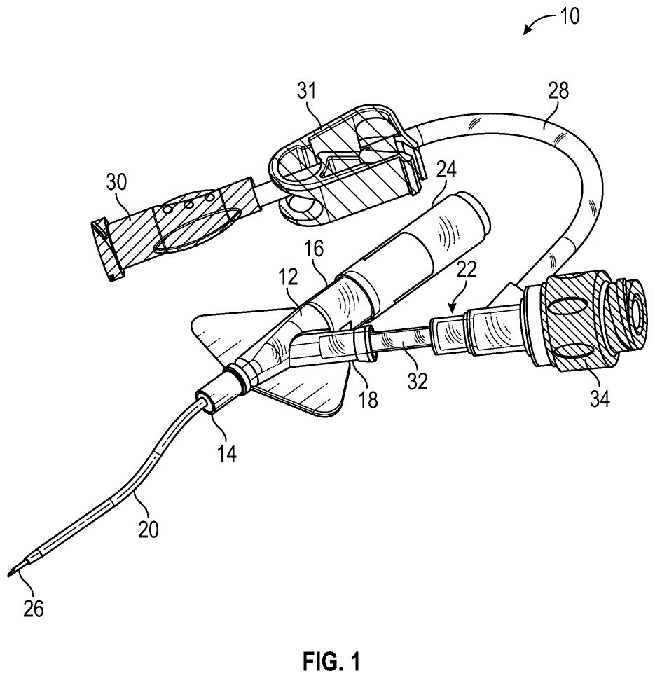

The present disclosure relates generally to a vascular access device having one or more phosphorescent features. In some embodiments, the phosphorescent features may facilitate operation of the vascular access device by a clinician without turning on a light in a room of a patient, thus reducing light disruption to the patient's sleep. The example phosphorescent features of FIGS. 1-5 and 7 are illustrated with striping.

Referring now to FIG. 1, in some embodiments, the vascular access device may include a catheter assembly 10. In some embodiments, the catheter assembly 10 may include a catheter hub 12, which may include a distal end 14, a proximal end 16, and a lumen extending through the distal end 14 and the proximal end 16. In some embodiments, the catheter hub 12 may include a side port 18 disposed between the distal end 14 and the proximal end 16. In some embodiments, the side port 18 may be in fluid communication with the lumen extending through the distal end 14 and the proximal end 16.

In some embodiments, the catheter assembly 10 may include a septum disposed within the lumen of the catheter hub 12 and configured to prevent fluid leakage out of the catheter hub 12. In some embodiments, the catheter assembly 10 may include a catheter 20 extending distally from the catheter hub 12. In some embodiments, the catheter 20 may include a peripheral-intravenous catheter (PIVC), a peripherally-inserted central catheter, a midline catheter, or another suitable catheter.

In some embodiments, the catheter assembly 10 may include an extension set 22 coupled to the catheter hub 12. In some embodiments, the catheter assembly 10 may include a needle hub 24 coupled to the proximal end 16 of the catheter hub 12. In some embodiments, the catheter assembly 10 may include an introducer needle 26 coupled to the needle hub 24 and extending through the catheter 20.

In some embodiments, the extension set 22 may extend proximally from the side port 18 and may include an extension tube 28 and a luer adapter 30 disposed at a proximal end of the extension tube 28. In some embodiments, the extension set 22 may include the extension tube 28 and a clamp 31 disposed on the extension tube 28. In some embodiments, the clamp 31 may be movable between a clamped position, in which fluid is prevented from travelling through the extension tube 28, and an unclamped position. In some embodiments, the extension tube 28 may extend from the side port 18. In other embodiments, the extension set 22 may include another extension tube 32, which may extend from the side port 18 and may be coupled to another luer adapter 34. In some embodiments, the extension tube 28 may extend from the other luer adapter 30.

In some embodiments, the one or more phosphorescent features of the catheter assembly 10 may facilitate operation of the vascular access device by a clinician without turning on a light in a room of a patient, thus reducing light disruption to the patient's sleep. In some embodiments, the one or more phosphorescent features may include features the clinician may contact to perform a procedure using the vascular access device, such as an infusion or blood draw.

In some embodiments, the clamp 31 may be phosphorescent, which may facilitate the clinician unclamping the clamp 31 during an infusion or blood draw procedure without turning on the light and disturbing the patient. In some embodiments, infusion may include a flushing procedure to flush the catheter 20. In some embodiments, the luer adapter 30 may be phosphorescent, which may facilitate attachment of a blood draw or infusion device to the luer adapter 30 without turning on the light and disturbing the patient. In some embodiments, the other luer adapter 34 may be phosphorescent, which may facilitate attachment of a blood draw or infusion device to the other luer adapter 34 without turning on the light and disturbing the patient. In some embodiments, the other luer adapter 34 may include a needleless access connector, which may include a valve therein. In some embodiments, the valve may be phosphorescent. In some embodiments, the phosphorescent material of the valve may include silicone and phosphorous, which is available in medical grades.

In some embodiments, the one or more phosphorescent features may include a phosphorescent material dispersed in a matrix of a body, which may include a light-transparent component. Additionally, or alternatively, the phosphorescent material may be disposed on a surface of the body as a coating. In some embodiments, when the one or more phosphorescent materials are exposed to an external light source, the phosphorescent material may absorb radiant energy from the external light source and electrons in the phosphorescent material become excited. In some embodiments, when the electrons are in an excited state, the phosphorescent material exhibits a glowing light discharge for a period of time determined by a decay of the electrons in the excited state. In some embodiments, the glowing light discharge may be at a wavelength within the visible light spectrum, including wavelengths of about 400 nanometers to about 700 nanometers, which may facilitate visualization by the clinician as the one or more phosphorescent features “glow in the dark.”

In some embodiments, the body in which the phosphorescent material may be dispersed may include a polymer material, such as polycarbonate, polypropylene, plastic, acrylonitrile butadiene styrene (ABS), or another suitable material. In some embodiments, the body of the phosphorescent feature may be molded from the polymer material, and the phosphorescent material may include a pigment based on zinc sulfide or a radioisotope to enhance luminosity. In some embodiments, the body may be molded from CGX 111274, a 45% loaded polycarbonate material that utilizes glow-in-the-dark pigments based on Strontium Oxide Aluminate chemistry.

In some embodiments, the phosphorescent material may be selected from materials known to those skilled in the art. Non-limiting examples of phosphorescent materials include non-oxide phosphors such as zinc sulfide phosphors, which may become excited quickly to attain maximum brightness. Zinc sulfides generally exhibit a glow light discharge for shorter periods of time than other phosphors. A zinc sulfide composition may be doped with at least one transition metal or rare earth metal to enhance photoluminescence excitation. For example, zinc sulfide doped with copper metal, i.e., ZnS:Cu, may require only a few seconds of ultraviolet or incident light exposure to provide a glow light discharge. Other zinc sulfide compositions may be configured to provide a glow light discharge with a particular hue. For instance, zinc sulfide doped with silver metal, i.e., ZnS:Ag, may provide a blue glow light discharge. Zinc sulfide doped with manganese metal, i.e., ZnS:Mn, may provide a green glow light discharge. These and other zinc sulfide compositions are known to those skilled in the art to provide a glow light discharge in response to photoluminescence excitation.

Other phosphors include long decay time phosphors such as oxide phosphors including, but not limited to, oxide ceramic phosphors. As in the zinc sulfide compositions, oxide ceramic phosphors may be doped, such as with a rare earth metal. These types of phosphors generally exhibit a long decay time. For example, an alkaline-earth metal oxide aluminate material may have longer glow light discharge time after exposure to radiant energy of the appropriate wavelength. These phosphors may be exposed to light for longer periods of time to achieve excitation to provide a longer and brighter glow light discharge relative to non-oxide phosphors. A typical alkaline-earth oxide aluminate may provide a glow light discharge still visible after about 24 hours. Suitable examples of non-oxide phosphors include, but are not intended to be limited to, strontium oxide aluminate doped with europium, strontium oxide aluminate doped with europium and dysprosium, and the like. Other suitable compositions are known to those skilled in the art.

In some embodiments, the catheter assembly 10 may include multiple phosphorescent features, which may increase light emitted from the catheter assembly 10, facilitating operation of the catheter assembly 10 by the clinician in the dark. For example, more than one of the luer adapter 30, the other luer adapter 34, and the clamp 31 could be phosphorescent. In some embodiments, portions of the catheter assembly 10 that that are less likely to be contacted by the clinician to perform a blood draw and/or infusion may not be phosphorescent to limit an amount of light emitted by the catheter assembly 10 to decrease a risk of disturbing the patient and the patient's sleep cycle.

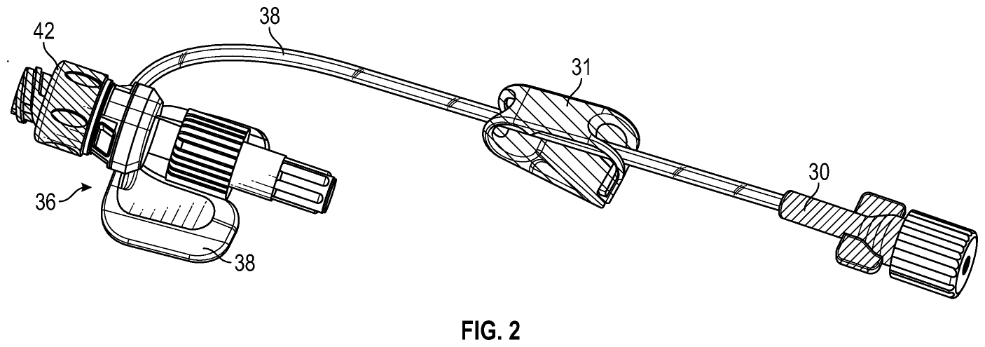

Referring now to FIG. 2, in some embodiments, a catheter hub stabilization device 36 may include a platform 38 and may be selectively coupled to the proximal end 16 of the catheter hub 12 (see FIG. 1) after removal of the needle hub 24 and the introducer needle 26. In these and other embodiments, the catheter assembly 10 may not include one or more wings 40. In some embodiments, a distal end 39 of the catheter hub stabilization device 36 may be inserted and/or threaded with the proximal end 16 of the catheter hub 12. In some embodiments, the extension set 22 may extend from the catheter hub stabilization device 36. In some embodiments, another luer adapter 42 may be coupled to a proximal end of the catheter hub stabilization device 36, which may include a fluid path therethrough in fluid communication with the lumen of the catheter hub 12 and the extension set 22.

In some embodiments, one or more of the following may be phosphorescent: the clamp 31, the luer adapter 30, and the other luer adapter 42. In some embodiments, the other luer adapter 42 may be phosphorescent, which may facilitate attachment of a blood draw or infusion device to the other luer adapter 42 without turning on the light and disturbing the patient. In some embodiments, the other luer adapter 42 may include a needleless access connector, which may include a valve therein. In some embodiments, the valve may be phosphorescent.

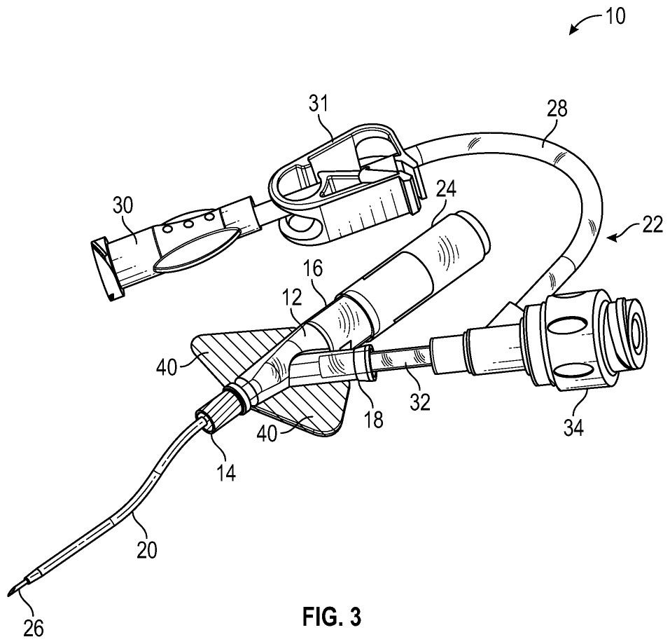

Referring now to FIG. 3, in some embodiments, a catheter hub stabilization device may include the one or more wings 40. In some embodiments, the one or more wings 40 may extend outwardly from the catheter hub. In some embodiments, the wings 40 may be phosphorescent, which may facilitate gripping by the clinician to insert the catheter assembly 10 into the vasculature of the patient without turning on the light and disturbing the patient. In these and other embodiments, the distal end 14 of the catheter hub 12 may be phosphorescent, which may help the clinician visualize how far to insert the catheter assembly 10 and facilitate insertion of the catheter assembly 10 into the vasculature of the patient without turning on the light and disturbing the patient. In some embodiments, the phosphorescent material of the platform 38 may include silicone and phosphorous, which is available in medical grades.

In some embodiments, portions of the catheter assembly 10 that that are less likely to be contacted by the clinician to insert the catheter assembly 10 into the patient may not be phosphorescent to limit an amount of light emitted by the catheter assembly 10 to decrease a risk of disturbing the patient and the patient's sleep cycle. For example, a portion of the catheter hub 12 between the wings 40 may not be phosphorescent.

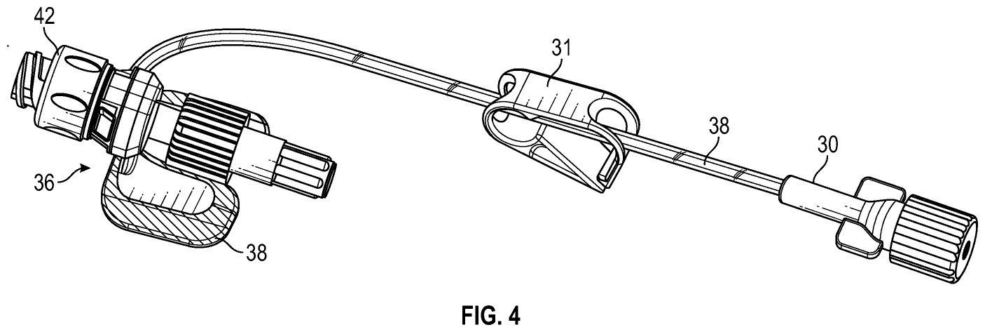

Referring now to FIG. 4, in some embodiments, the platform 38 may be phosphorescent, which may facilitate location of the catheter hub stabilization device 36 by the clinician for coupling to the proximal end 16 of the catheter hub 12. In some embodiments, an edge of the platform 38 or other catheter stabilization device may be phosphorescent, which may limit an amount of light emitted by the catheter assembly 10 to decrease a risk of disturbing the patient and the patient's sleep cycle. In some embodiments, the phosphorescent material of the platform 38 may include silicone and phosphorous, which is available in medical grades.

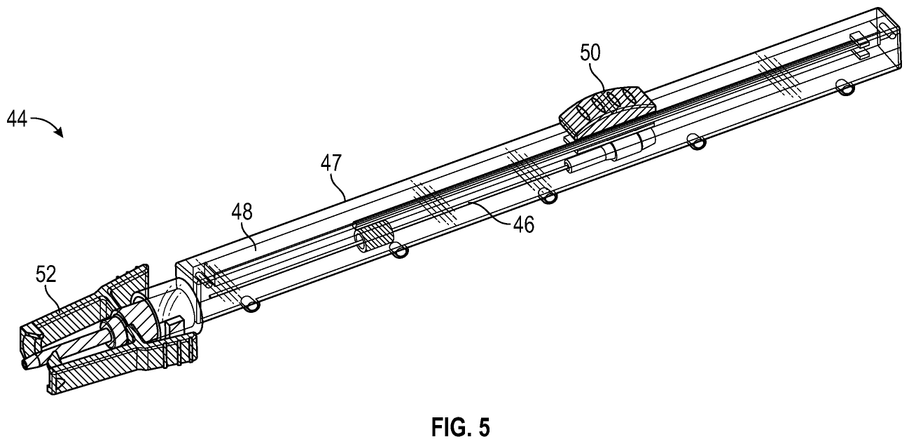

Referring now to FIG. 5, in some embodiments, the vascular access device may include an instrument advancement device 44, which may be configured to advance an instrument 46 distally through the catheter assembly 10 (see FIGS. 1-4). In some embodiments, the instrument advancement device 44 may be similar or identical to the PIVO™ device, previously available from VelanoVascular™, in terms of one or more features and/or operation. In some embodiments, the instrument advancement device 44 may include a housing 47, which may include a slot 48. In some embodiments, the instrument advancement device 44 may include an advancement tab 50 disposed within the slot 48. In some embodiments, the instrument advancement device 44 may include the instrument 46 disposed within the housing 47 and coupled to the advancement tab 50. In some embodiments, the instrument 46 may include a guidewire or a catheter.

In some embodiments, the instrument advancement device 44 may include a distal connector 52 configured to coupled to the catheter assembly 10. In some embodiments, the distal connector 52 may include two prongs configured to clasp the proximal end 16 of the catheter hub 12, and a central portion of the distal connector 52 may be inserted into the lumen of the catheter hub. In some embodiments, the instrument advancement device 44 may include a support feature 54 disposed within the housing 47 and configured to reduce buckling of the instrument 46. In some embodiments, the support feature may include a tubular structure or another suitable structure.

In some embodiments, the distal connector 52 may be phosphorescent, which may facilitate attachment of the instrument advancement device 44 to the other luer adapter 42 (see, e.g. FIG. 4) and/or the proximal end 16 of the catheter hub 12 without turning on the light and disturbing the patient. In some embodiments, the advancement tab 50 may be phosphorescent, which may facilitate movement of the advancement tab 50 along the slot 48 to advance the instrument 46 through the catheter assembly into the patient and/or retract the instrument 46 without turning on the light and disturbing the patient. In some embodiments, the support feature 54 may be phosphorescent, which may facilitate visualization of the housing 47 for gripping by the clinician. In these and other embodiments, the housing 47 may be translucent or transparent.

In some embodiments, the instrument advancement device 44 may include multiple phosphorescent features, which may increase light emitted from the instrument advancement device 44, facilitating operation of the catheter assembly 10 by the clinician in the dark. For example, more than one of the distal connector 52, the advancement tab 50, and the support feature 54 could be phosphorescent. In some embodiments, portions of the instrument advancement device 44 that are not needed to connect the instrument advancement device 44 to the catheter assembly 10 and/or advance the instrument 46 may not be phosphorescent to limit an amount of light emitted by the instrument advancement device 44 to decrease a risk of disturbing the patient and the patient's sleep cycle.

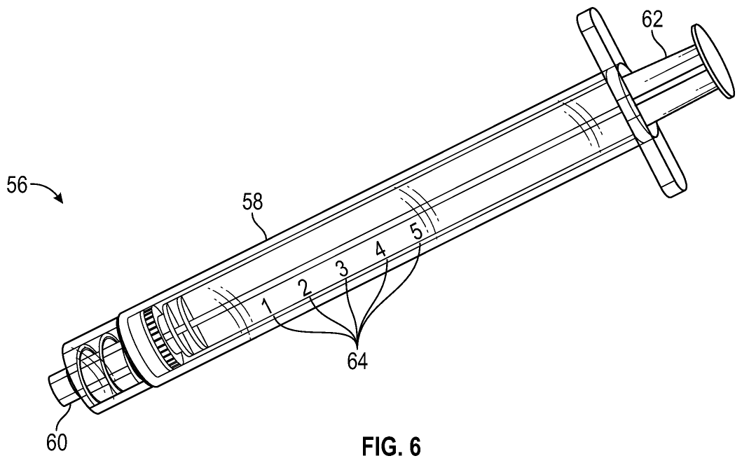

Referring now to FIG. 6, in some embodiments, the vascular access device may include a syringe 56 configured to be coupled to the catheter assembly 10 to draw blood and/or flush the catheter assembly 10. In some embodiments, the syringe 56 may include a barrel 58, which may include a distal end 60 configured to couple to the catheter assembly. In some embodiments, the syringe 56 may include a plunger 62 configured to depress within the barrel. In some embodiments, the syringe 56 may include a phosphorescent feature, which may facilitate operation of the syringe 56 in the dark or without turning on the light in the patient's room in order to perform an infusion, flush, or blood draw. In some embodiments, one or more stickers 64 may be disposed on the barrel 58 and may include phosphorescent text indicating a volume. For example, the numbers 1-5 may correspond to volumes (e.g., mL) in the syringe 56 when the plunger 62 is aligned with a particular number.

In some embodiments, the phosphorescent feature of the syringe 56 may include the phosphorescent material dispersed in a matrix of the barrel 58, which may include the light-transparent component. Additionally or alternatively, the phosphorescent material may be disposed on a surface of the barrel 58 as a coating.

In some embodiments, the catheter assembly 10 of FIGS. 1-4, the instrument advancement device 44 of FIG. 5, and the syringe 56 of FIG. 6 may include other phosphorescent features than those illustrated. For example, the instrument advancement device 44 may include a luer, a clamp, and/or the instrument that is phosphorescent, which may aid in advancing the instrument distally. As another example, the catheter assembly 10 may include one or more phosphorescent caps, which may aid in removal when the patient's light is off.

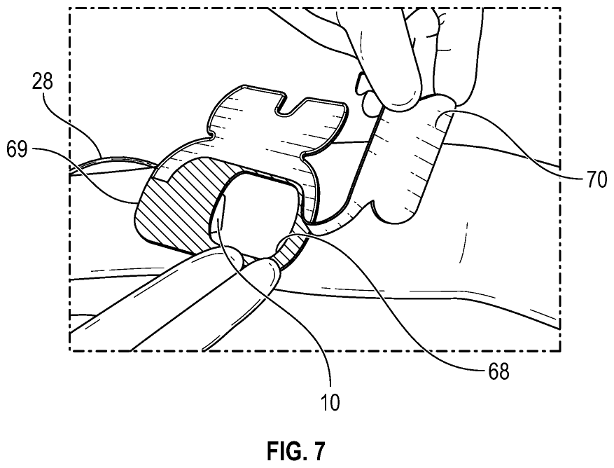

Referring now to FIG. 7, in some embodiments, the phosphorescent material may be integrated into a dressing 66 configured to secure the catheter assembly 10. In some embodiments, the phosphorescent material may be dispersed in a matrix of the dressing 66, which may include the light-transparent component. Additionally or alternatively, the phosphorescent material may be disposed on a surface of the dressing 66 as a coating.

In some embodiments, the phosphorescent material may surround a window 68, which may include clear plastic, and facilitate appropriate placement of the dressing 66 with respect to the inserted catheter assembly 10. In these and other embodiments, the phosphorescent material may be disposed on a border or edge 69 of the dressing 66 and/or disposed from the edge 69 to the window 68, which may facilitate visualization of the edge and/or the window 68 by the clinician when the catheter assembly 10 is inserted into an arm of the patient. In some embodiments, a cover 70 configured to facilitate a sterile dressing may be removed from the dressing 66 when the dressing 66 is in use. In some embodiments, the dressing 66 may include a phosphorescent sticker in place of the phosphorescent material integrated into the dressing 66. In some embodiments, one or more features of the catheter assembly 10, the instrument advancement device 44, and the syringe 56 may be included in a kit, which may also include a small flashlight or UV light.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A catheter assembly, comprising:
a catheter hub, comprising a distal end, a proximal end, a lumen extending through the distal end and the proximal end, and a side port disposed between the distal end and the proximal end;
a catheter extending distally from the catheter hub;
an extension set coupled to the catheter hub;
a catheter hub stabilization device coupled to the catheter hub;
a needle hub coupled to the proximal end of the catheter hub; and
an introducer needle coupled to the needle hub and extending through the catheter,
wherein the extension set, the catheter hub, or the catheter hub stabilization device is phosphorescent.

2. The catheter assembly of claim 1, wherein the extension set extends proximally from the side port, wherein the extension set comprises an extension tube and a luer adapter disposed at a proximal end of the extension tube, wherein the luer adapter is phosphorescent.

3. The catheter assembly of claim 1, wherein the extension set extends proximally from the side port, wherein the extension set comprises an extension tube and a clamp disposed on the extension tube, wherein the clamp is phosphorescent.

4. The catheter assembly of claim 3, wherein the extension set extends proximally from the side port, wherein the extension set comprises an extension tube and a luer adapter disposed at a proximal end of the extension tube, wherein the extension set further comprises another extension tube extending from the side port and coupled to another luer adapter, wherein the extension tube extends from the other luer adapter, wherein the other luer adapter is phosphorescent.

5. The catheter assembly of claim 4, wherein the luer adapter and the clamp are phosphorescent.

6. The catheter assembly of claim 1, wherein the distal end of the catheter hub is phosphorescent.

7. The catheter assembly of claim 1, wherein the catheter stabilization device is phosphorescent.

8. The catheter assembly of claim 1, wherein the catheter stabilization device comprises a wing extending outwardly from the catheter hub, wherein the wing is phosphorescent.

9. The catheter assembly of claim 1, wherein the catheter stabilization device comprises a platform selectively coupled to the catheter hub, wherein the platform is phosphorescent.

10. The catheter assembly of claim 1, wherein the extension set, the catheter hub, or the catheter hub stabilization device comprises a phosphorescent material as a coating or within a matrix.

11. An instrument advancement device configured to advance a catheter distally through a catheter assembly, the instrument advancement device comprising:
a housing, comprising a slot;
an advancement tab disposed within the slot;
an instrument disposed within the housing and coupled to the advancement tab;
a distal connector configured to coupled to the catheter assembly; and
a support feature disposed within the housing and configured to reduce buckling of the instrument, wherein the support feature, the advancement tab, or the distal connector is phosphorescent.

12. The instrument advancement device of claim 11, wherein the advancement tab is phosphorescent.

13. The instrument advancement device of claim 12, wherein the distal connector is phosphorescent.

14. The instrument advancement device of claim 11, wherein the distal connector is phosphorescent.

15. The instrument advancement device of claim 11, wherein the support feature is phosphorescent.

16. A syringe configured to be coupled to a catheter assembly to draw blood, the syringe comprising:
a barrel, comprising a distal end configured to couple to the catheter assembly; and
a plunger configured to depress within the barrel; and
a phosphorescent feature, comprising a phosphorescent material molded into the barrel or a sticker on the barrel comprising phosphorescent text indicating a volume.

17. The syringe of claim 16, wherein the phosphorescent feature comprises the phosphorescent material molded into the barrel.

18. The syringe of claim 16, wherein the phosphorescent feature comprises the sticker.

19. The syringe of claim 16, wherein the phosphorescent text comprises a plurality of numbers.
